(19) United States
(12) Patent Application Publication
(10) Pub. No.: US 2024/0257979 A1
(43) Pub. Date: Aug. 1, 2024

(54) MACHINE LEARNING ENABLED PROGNOSIS OF PATIENT MORTALITY

(71) Applicant: City of Hope, Duarte, CA (US)

(72) Inventors: Finly Zachariah, Duarte, CA (US); Laura Roberts, Duarte, CA (US); Lorenzo Rossi, Duarte, CA (US)

(21) Appl. No.: 18/425,689

(22) Filed: Jan. 29, 2024

Related U.S. Application Data
(60) Provisional application No. 63/482,277, filed on Jan. 30, 2023.

(51) Int. Cl. G16H 50/30; G16H 10/20

(52) U.S. Cl. CPC G16H 50/30; G16H 10/20

(57) ABSTRACT

A method for machine learning enabled mortality prognosis may include training, based on a set of labeled training samples, a mortality prognosis model to determine a risk of patient mortality within a given timeframe such as one week, two weeks, three weeks, one month, three months, six months, nine months, one year, 18 months, two years, three years, five years, and/or the like. The trained mortality prognosis model may be applied to determine, based on a health record of a patient, a risk of mortality for the patient within the given timeframe. A treatment plan for the patient may be determined based on the risk of mortality for the patient within the given timeframe. Related systems and computer program products are also provided.

### CROSS REFERENCE TO RELATED APPLICATIONS

This application claims priority to U.S. Provisional Patent Application No. 63/482,277, filed on Jan. 30, 2023, and entitled “Machine Learning Enabled Prognosis of Patient Mortality,” the disclosures of this application are incorporated herein by reference.

### TECHNICAL FIELD

The subject matter described herein relates generally to machine learning and more specifically to a machine learning based prognosis model for assessing patient mortality.

### INTRODUCTION

Patients and families rely on clinicians to provide transparent and precise prognostic information to make informed, value-based choices about end-of-life care. However, physicians often overestimate survival or are reticent to discuss prognosis and end-of-life preferences owing to perceived patient distress, rapidly progressive science, and lack of prognostic confidence. This overestimation may result in unwanted care and overuse of healthcare services near the end of life. Within the oncology population, there remains high use of intensive care and chemotherapy and underuse of hospice care near the end of life, resulting in billions of dollars in unnecessary healthcare expenses. As such, improving prognostic confidence and facilitating alignment between patient values and therapeutic delivery represents an important value proposition for patients, caregivers, clinicians, and payers.

### SUMMARY

Embodiments of the present disclosure can include a computer-implemented method including the steps of: training, based at least on a set of labeled training samples, a mortality prognosis model to determine a risk of patient mortality within a given timeframe; applying the trained mortality prognosis model to determine, based at least on a health record of a patient, a risk of mortality for the patient within the given timeframe; and determining, based at least on the risk of mortality for the patient within the given timeframe, a treatment plan for the patient.

In some embodiments, the mortality prognosis model can include a machine learning model. For example, the mortality prognosis model can be based on one or more of a logistic regression, a tree ensemble, a support vector machine, a k-nearest neighbor clustering model, a shallow neural network, or a deep neural network. The trained mortality prognosis model can be configured to determine, based at least on a plurality of features extracted from the health record of the patient, the risk of mortality for the patient within the given timeframe. Optionally, the plurality of features can include one or more clinical attributes including at least one of a count of normal lab results, an observation duration, a minimum level of albumin, a low reference for latest albumin level, a standard deviation in weight, a high reference for the latest albumin level, a linear trend in weight, a diagnosis, an approximate initial body mass index (BMI), a minimum white blood cell count (WBC), a low reference for latest hemoglobin level, a low reference for a latest percentage of lymphocytes, age, a low reference for a latest level of alkaline phosphatase (ALP), a linear trend in body mass index (BMI), a minimum level of hemoglobin, a standard deviation in body mass index (BMI), a low reference for latest level of lactate dehydrogenase (LDH), an approximate initial level of alkaline phosphatase (ALP), a diagnosis, a medication, a performance status, utilization, notes, and Logical Observation Identifiers Names and Codes (LOINC).

In some embodiments the health record can be an electronic health record of the patient. The set of labeled training samples can include a plurality of training samples, and each training sample of the plurality of training samples can be associated with a patient. Optionally, each training sample of the plurality of training samples can include one or more demographic characteristics, laboratory test results, flowsheets, and diagnoses of a corresponding patient. One or more training samples of the plurality of training samples can further include a ground truth annotation of an observation of the corresponding patient being deceased or alive at an end of the given timeframe.

The method can also include the step of generating the set of labeled training samples to include no more than a threshold quantity of training samples associated with patients who die within a threshold quantity of time. The given timeframe can be one week, two weeks, three weeks, one month, three months, six months, nine months, one year, 18 months, two years, three years, or five years.

The treatment plan for the patient can be determined to include end-of-life care where the risk of mortality for the patient satisfies a threshold value. The treatment plan for the patient can be further determined based on one or more patient preferences, patient values, and patient priorities. In some embodiments the treatment plan for the patient is further determined based on one or more patient preferences, patient values, and patient priorities where the risk of mortality for the patient satisfies a threshold value. The treatment plan for the patient can be determined to include one or more clinical trials where the risk of mortality for the patient satisfies a threshold value. The treatment plan for the patient can be determined to include end-of-life care where the risk of mortality for the patient within a first timeframe satisfies a first threshold value, and wherein the treatment plan for the patient is determined to include one or more clinical trials where the risk of mortality for the patient within the first timeframe and/or a second timeframe satisfies a second threshold value.

The treatment plan can be reevaluated for the patient where the risk of mortality for the patient satisfies a threshold value. Re-evaluating the treatment plan can include applying the trained mortality prognosis model to determine the risk of mortality for the patient at a first timepoint before applying the trained mortality prognosis model to determine the risk of mortality for the patient at a second timepoint, and adjusting the treatment plan where a difference in the risk of mortality for the patient at the first timepoint and the second timepoint exceeds a threshold value.

Systems, methods, computer program products, and articles of manufacture can be provided for the same.

### DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a prognosis system, in accordance with some example embodiments;

FIG. 2A depicts a table illustrating various variables of an example patient cohort, in accordance with some example embodiments;

FIG. 2B depicts a table illustrating a comparison of an instance of a mortality model and a conventional mortality prognostic tool for an example patient cohort, in accordance with some example embodiments;

FIG. 2C depicts a table illustrating another comparison of an instance of a mortality model and a conventional mortality prognostic tool for an example patient cohort, in accordance with some example embodiments;

FIG. 3 depicts tables illustrating a receiver operating characteristic (ROC) curve, a precision recall curve (PRC), and a survival curve for an instance of a mortality model and a conventional mortality prognostic tool, in accordance with some example embodiments;

FIG. 4 depicts scatterplots illustrating the positive predictive value (PPV) and sensitivity of an instance of a mortality model and a conventional mortality prognostic tool, in accordance with some example embodiments;

FIG. 5 depicts a table illustrating examples of predictive features for an instance a mortality model, in accordance with some example embodiments;

FIG. 6 depicts a flowchart illustrating an example of a process for machine learning enabled prognosis of patient mortality, in accordance with some example embodiments;

FIG. 7 depicts a block diagram illustrating an example system integrated with a prognosis system; and

FIG. 8 depicts a block diagram illustrating an example of a computing system, in accordance with some example embodiments.

When practical, similar reference numbers denote similar structures, features, or elements.

### DETAILED DESCRIPTION

Reliable and consistently applied prognostic tools in oncology may enhance prognostic confidence, increase prognostic authority, and improve the clarity and strength of medical recommendations for and against therapies. Many prognostic scales have been studied in oncology, some with more administrative burden. One conventional mortality prognostic tool is the surprise question (SQ), which asks clinicians whether it would surprise them if a patient died within a particular time frame. The surprise question has been used most commonly with a one-year time frame but also with time frames between one week and six months with varying performance. The surprise question has demonstrated moderately superior performance in oncology populations when compared with heart failure, kidney failure, and diagnoses examined in other studies. However, conventional mortality prognostic tools, such as the surprise question, are imprecise and prone to errors such as, for example, a tendency to over- and/or underestimate survival.

In some example embodiments, a prognostic engine may apply, to one or more electronic health records (EHR) of a patient, a mortality prognosis model trained to determine the patient's risk of mortality within a specific timeframe (e.g., one week, two weeks, three weeks, one month, three months, six months, nine months, one year, 18 months, two years, three years, five years, and/or the like). In some cases, the mortality prognosis model may be implemented using one or more (semi) supervised machine learning models including, for example, logistic regression, tree ensembles such as random forest and gradient boosted trees, support vector machines, k-nearest neighbors clustering model, shallow or deep neural networks, and/or the like. When compared to conventional mortality prognostic tools, such as the surprise question, the machine learning based mortality prognosis model achieved significantly superior prognostic performance. For example, in a patient cohort with a 15% prevalence of 3-month mortality and 30% sensitivity for the conventional mortality prognosis tool and the machine learning based mortality prognosis model, the positive predictive value (PPV) of the conventional mortality prognosis tool is merely 34.8% (95% confidence interval, 30.1%-39.5%) whereas the positive predictive value (PPV) of the machine learning based mortality prognosis model is 60.0% (95% confidence interval, 53.6%-66.3%). Moreover, the area under the receiver operating characteristic curve of the machine learning based mortality prognosis model is 81.2% (95% confidence interval, 79.1%-83.3%).

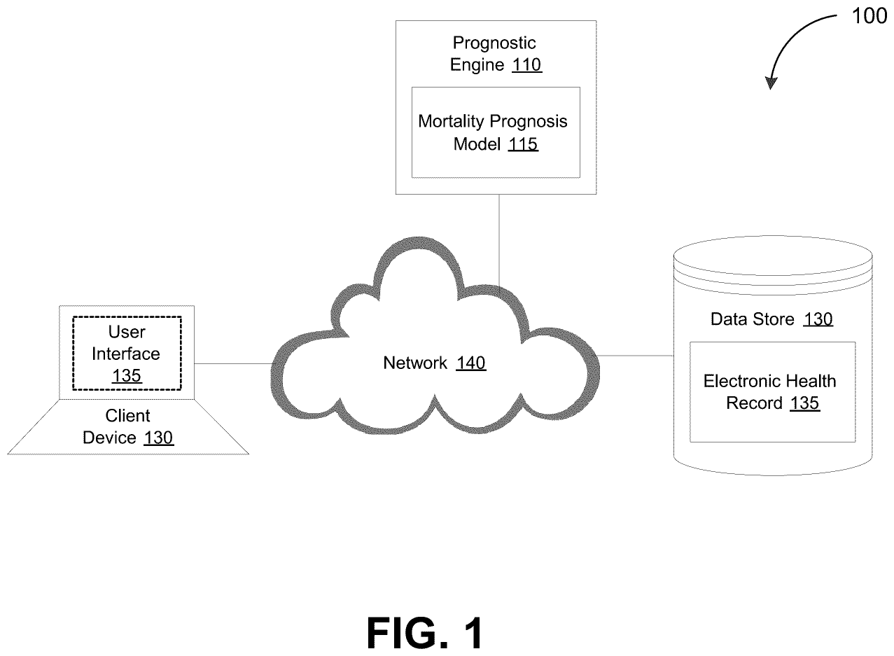

FIG. 1 depicts a system diagram illustrating an example of a prognosis system 100, in accordance with some example embodiments. Referring to FIG. 1, the prognosis system 100 may include a prognostic engine 110, a client device 120, and a data store 130. As shown in FIG. 1, the prognostic engine 110, the client device 120, and the data store 130 may be communicatively coupled via a network 140. The client device 120 may be a processor-based device including, for example, a workstation, a desktop computer, a laptop computer, a smartphone, a tablet computer, a wearable apparatus, and/or the like. The data store 130 may be a relational database, a non-structured query language (NoSQL) database, an in-memory database, a graph database, a key-value store, a document store, an operational database, an analytical database, and/or the like. The network 140 may be a wired network and/or a wireless network including, for example, a local area network (LAN), a virtual local area network (VLAN), a wide area network (WAN), a public land mobile network (PLMN), the Internet, and/or the like.

In some example embodiments, the prognostic engine 110 may apply a mortality prognosis model 115 to determine, based at least on one or more electronic health records (EHR) 135 of a patient from the data store 130, the patient's risk of mortality within a specific timeframe (e.g., one week, two weeks, three weeks, one month, three months, six months, nine months, one year, 18 months, two years, three years, five years, and/or the like). The mortality prognosis model 115 may be implemented using one or more machine learning models including, for example, logistic regression, tree ensembles such as random forest and gradient boosted trees, support vector machines, k-nearest neighbors clustering model, shallow or deep neural networks, and/or the like.

In some example embodiments, the mortality prognosis model 115 may be trained in a supervised manner based on a set of labeled training samples before being evaluated on a set of labeled evaluation samples. In this context, a labeled sample (e.g., a labeled training sample or a labeled valuation sample) refers to a sample that is associated with a ground truth annotation indicating the correct (or observed) mortality prognosis for a corresponding set of patient features. For example, each training sample in the training set and each evaluation sample in the evaluation set may be associated with a patient and include one or more features collected from the patient's electronic health records such as demographic characteristics, laboratory test results, flowsheets, and diagnoses as well as a ground truth annotation of an observation of the patient being deceased or alive at a particular timepoint (e.g., one week, two weeks, three weeks, one month, three months, six months, nine months, one year, 18 months, two years, three years, five years, and/or the like).

In some cases, the training set and the evaluation set may undergo at least some preprocessing including, for example, the exclusion of patients without a threshold quantity of data. For instance, for inclusion in the training set or the evaluation set, a patient may be required to have a minimal quantity of data such as at least two encounters and, for living patients, a completed visit documented in an electronic health record at least 1 year post prediction date to avoid observations with potential missing death information. To limit the risk of data leakage, dates of prediction may be selected to exclude encounters within a threshold quantity of time (e.g., seven days) from death. To avoid over representing observations in which the corresponding patient die within a threshold quantity of time (e.g., thirty days), the mortality prognosis model 115 may be trained using no more than a threshold quantity of training samples associated with patients who die within a threshold quantity of time, thus avoiding to train the mortality prognosis model 115 with a disproportionate number of near-term deceased patients.

In some example embodiments, to determine a patient's risk of mortality within a certain timeframe (e.g., one week, two weeks, three weeks, one month, three months, six months, nine months, one year, 18 months, two years, three years, five years, and/or the like), the mortality prognosis model 115 may operate on a variety of features extracted from the electronic health records of the patient. In some cases, the electronic health record of the patient may include time series data, such as laboratory test results, flowsheet data, and/or the like. One or more hand-crafted features may be extracted from this time series data including for example, the time series of laboratory test results and flowsheet data in a temporal window (e.g., a 180-day temporal window) preceding each prediction. Missing values for some features may be imputed in limited instances where the imputed values are obvious at least because the mortality prognosis model 115, when implemented as a tree-based classifiers, is capable of handling missing data. Table 1 below lists some example clinical variables used by the mortality prognosis model 115 including, for example, age, sex, race, and body mass index (BMI) as well as other features extracted from laboratory test and flowsheet time series data.

TABLE 1

| category | clinical variable | feature |
|---|---|---|
| Laboratory | Lymphocite %, Albumin (Blood), | count, median, minimum, maximum, |
| Tests | Calcium (Blood), WBC, LDH (Blood), | standard deviation, slope, intercept, |
| | Hemoglobin (Blood), Platelet Count, | early/late difference to lower/upper |
| | Alkaline Phosphatase, Creatinine | reference, % normal results, |
| | (Blood), Bilirubin (Blood), RBC, B12 | % very abnormal results |
| | (Serum), Segmented Neutrophil % | % lab late night orders |
| Flowsheet | weight, BMI | count, median, minimum, maximum, |
| | | standard deviation, slope, intercept, |
| | | early/late difference to l/u ref. |
| Demographic | age, gender, race | value |
| Diagnoses | ICD-9 codes | embedding aggregations |

In some example embodiments, the features extracted from the electronic health record of the patient, which includes features extracted from laboratory test and flowsheet time series data, may be encoded such that the mortality prognosis mode 115 operates on an embedding of the features. For example, the features associated with the diagnoses may include aggregations of word2vec embeddings of the International Classification of Diseases, Ninth Revision codes. A portion of the observations may be used for retrospective evaluation based on a temporal split at a single time point to mimic deployment in the real world, in which past observations are used to train the mortality prognosis model 115 to predict in the present.

Once the hyperparameters of the mortality prognosis model 115 have been tuned, for example, via cross-validation and retrospective evaluation, a version of the mortality prognosis model 115 may be retrained and deployed. For example, when implemented as a tree-based classifier, the trained mortality prognosis model may include an ensemble of decision trees. In some cases, the prognostic engine 110 may apply the mortality prognosis model 115 to make batches of predictions from observations automatically queried once from the data store 130, with the predictions being triggered the presence of new results from laboratory tests.

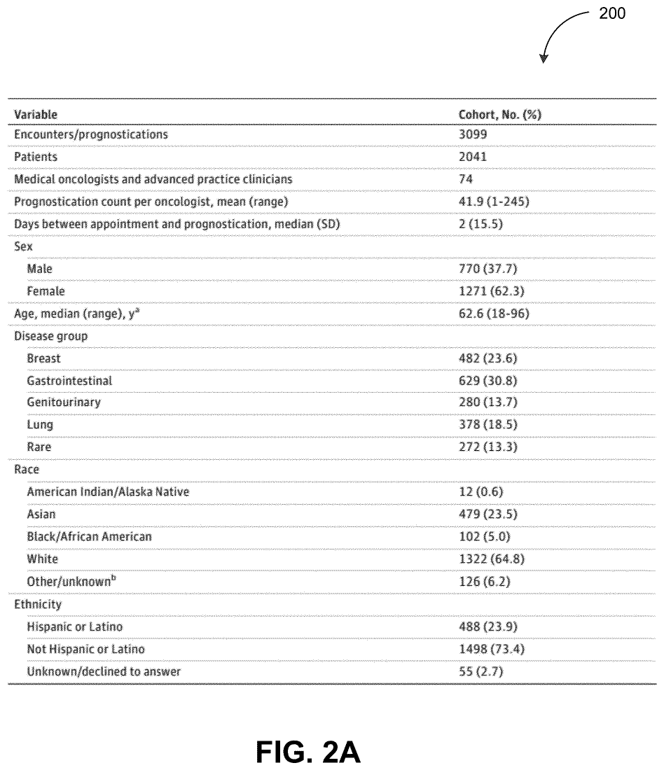

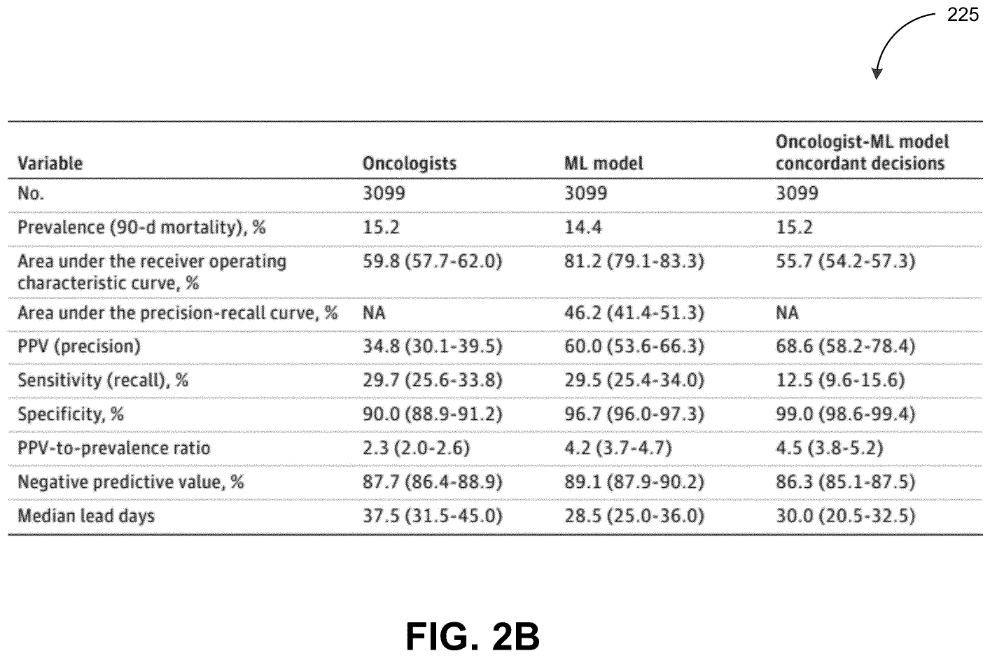

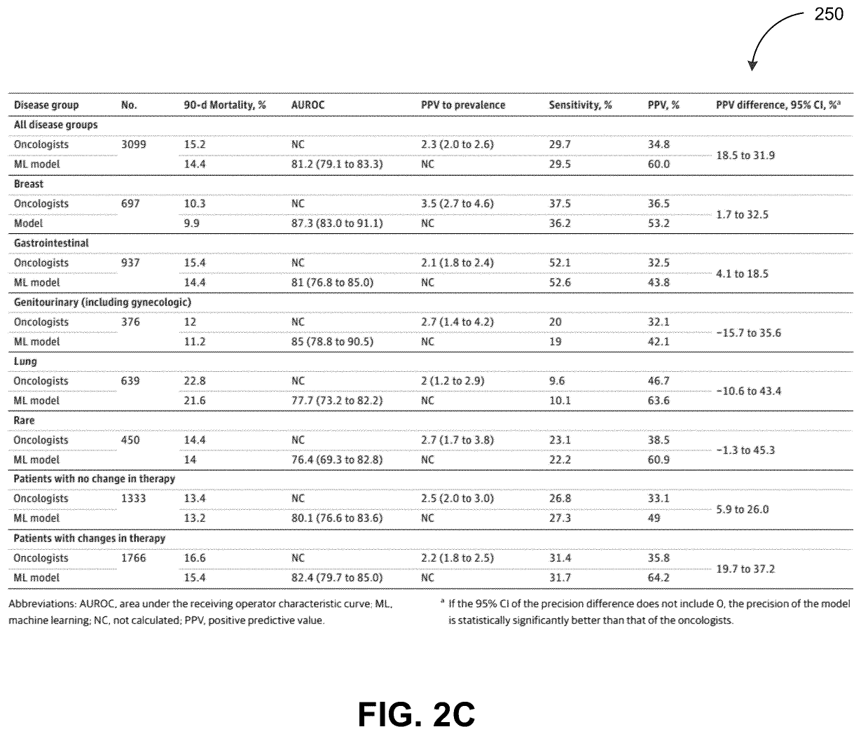

The prognostic performance of the trained mortality prognosis model 115 may be compared against that of conventional mortality prognosis tools such as surprise question (e.g., three-month surprise question) for a patient cohort that includes 3099 predictions from both the trained mortality prognosis model 115 and the conventional mortality prognosis tool (e.g., the three-month surprise question) for 2041 patients with advanced cancer. FIG. 2A depicts a table 200 illustrating various variables of the patient cohort including, for example, gender, age, disease group, race, and ethnicity. The prognostic study compared the performance of three-month mortality predictions made by the conventional mortality prognostic tool (e.g., the three-month surprise question) and the trained mortality prognosis model 115 for patients with metastatic solid tumors. The predictions made using the conventional mortality prognostic tool (e.g., the three-month surprise question) were paired with the closest prediction made by the trained mortality prognosis model 115 within the preceding 30 days. The primary outcome of the study was an in-depth comparison between the performance of the conventional mortality prognostic tool (e.g., the three-month surprise question) and the trained mortality prognosis model 115 in predicting three-month mortality for a population of patients with metastatic solid tumors.

Referring again to the table 200 shown in FIG. 2A, the patient cohort included 3099 pairs of predictions for 2041 unique patients. The mortality prediction from the trained mortality prognosis model 115 may include a score between 0 and 1 (with a value close to 1 indicative of a high mortality risk) whereas the conventional mortality prognostic tool provided a binary mortality prediction (e.g., yes or no). Thus, in order to compare the predictions from made by the mortality prognosis model 115 and the conventional mortality prognostic tool (e.g., the three-month surprise question), a threshold may be set to convert the risk scores output by the mortality prognosis model 115 into a binary output matching that of the conventional mortality prognostic tool (e.g., flag patients with an above threshold score (e.g., score >0.5) as at risk of three-month mortality). The decision threshold may be set to match the sensitivity of the mortality prognosis model 115 to that of the conventional mortality prognostic tool. Accordingly, the prognostic performance of the mortality prognosis model 115 and the conventional mortality prognostic tool may be compared based on their respective positive predictive value (PPV) or precision, which is the ratio of correct predictions over the total number of predictions made. The magnitude of the positive predictive value (PPV) depends on prevalence. Accordingly, the comparison of prognostic performance further takes into account the ratio between positive predictive value (PPV) and prevalence. In a scenario of pure random guessing, the ratio between positive predictive value (PPV) and prevalence would asymptotically converge to 1. The positive predictive value (PPV) to prevalence ratio may be a helpful metric for comparing the performance of binary predictions (surprised or not surprised) across subpopulations with different prevalence. As noted, in a situation of random guessing, this ratio would tend to 1. Omitting this ratio may conflate results close to random guessing (e.g., with positive predictive value (PPV) close to prevalence) with good performance.

Confidence intervals, such as the 95% confidence intervals of the metrics, may be computed via bootstrapping. The prognostic performance of the mortality prognosis model 115 and the conventional mortality prognostic tool (e.g., the three-month surprise question) may be compared within disease groups based on the 95% confidence intervals of the difference between the respective positive predictive values (PPV) at least because the 95% confidence intervals of the two positive predictive values overlapped. A 95% confidence interval of the difference above 0 means the mortality prognosis model 115 outperforms the conventional mortality prognostic tool with statistical significance.

In some cases, the performance of the mortality prognosis model 115 and the conventional mortality prognostic tool can also be characterized performance through sensitivity (or recall), specificity, and median lead days. Lead days in this context may be defined as the number of days between a correct mortality prediction and the actual date of death. For the mortality prognosis model 115, performance may also be assessed by computing the area under the receiver operating characteristic curve (AUROC) and area under the precision-recall curve. FIG. 2B depicts a table 225 illustrating the results for the conventional mortality prognostic tool and the mortality prognosis model 115 while FIG. 2C depicts a table 250 illustrating the results with stratified evaluations over disease groups and changes in systemic therapy.

As noted, the prognostic performance of the mortality prognosis model 115 was compared to that of a conventional mortality prognostic tool (e.g., the three-month surprise question) based on 3099 pairs of three-month mortality predictions 2041 patients (1271 [62.3%] women; 770 [37.7%] men) with a median age of 62.6 (range, 18-96) years at the time of prediction made by the conventional mortality prognostic tool. The median lag between the output of the conventional mortality prognostic tool and that of the mortality prognosis model 115 was three days, with 75% of model predictions made within eight days of the corresponding prediction by the conventional mortality prognostic tool. A decision threshold was set so that the sensitivity of the mortality prognosis model 115 matched the 30% sensitivity of the conventional mortality prognostic tool. The results shown in table 225 of FIG. 2B and table 250 of FIG. 2C indicate that the mortality prognosis model 115 outperformed the conventional mortality prognostic tool in aggregate (PPV, 60.0%; 95% CI, 53.6%-66.3% vs 34.8%; 95% CI, 30.1%-39.5%; P<0.001) (Table 2) and within breast (PPV difference, 16.7%; 95% CI, 1.7%-32.5%; P=0.03) and gastrointestinal (PPV difference, 11.3%; 95% CI, 4.1%-18.5%; P=0.002) disease subgroups (ie, 95% CI PPV difference >0) (Table 3). The positive predictive value (PPV) difference was not statistically significant within genitourinary (including gynecologic), lung, and rare cancer groups. Where the mortality prognosis model 115 and the conventional mortality prognostic tool arrived at a same prediction, the positive predictive value (PPV) of the conventional mortality prognostic tool increased from 34.8% (95% CI, 30.1%-39.5%) to 68.6% (95% CI, 58.2%-78.4%), with a decrease in sensitivity to 12.5% (Table 2).

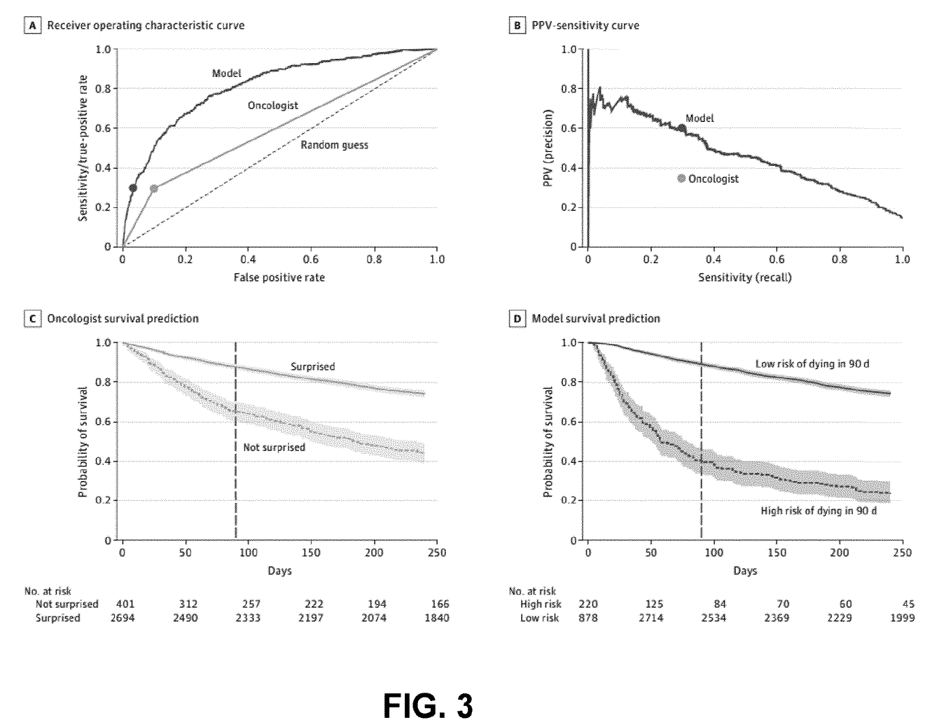

FIG. 3(A) depicts a receiving operating characteristic curve while FIG. 3(B) depicts a positive predictive value (PPV) sensitivity or PRC for the mortality prognosis model 115 and the conventional mortality prognostic tool (e.g., the three-month surprise question). The model's area under the receiving operating characteristic curve (AUROC) is 81.2% (95% CI, 79.1%-83.3%) whereas the area under the receiving operating characteristic cure (AUROC) for the conventional mortality prognostic tool is 59.8% (95% CI, 57.7%-62.0%). As the comparison shown in FIGS. 3(A) and (B) indicates, the model operating at the same sensitivity level of the conventional mortality prognostic tool achieved lower false-positive rates and higher positive predictive values (PPV). FIG. 3(C) depicts the survival curves for the conventional mortality prognostic tool while the survival curves for the mortality prognosis model 115 are shown in FIG. 3(D). As shown in FIGS. 3(C) and (D), the mortality prognosis model 115 demonstrates better discriminative ability than the conventional mortality prognostic tool.

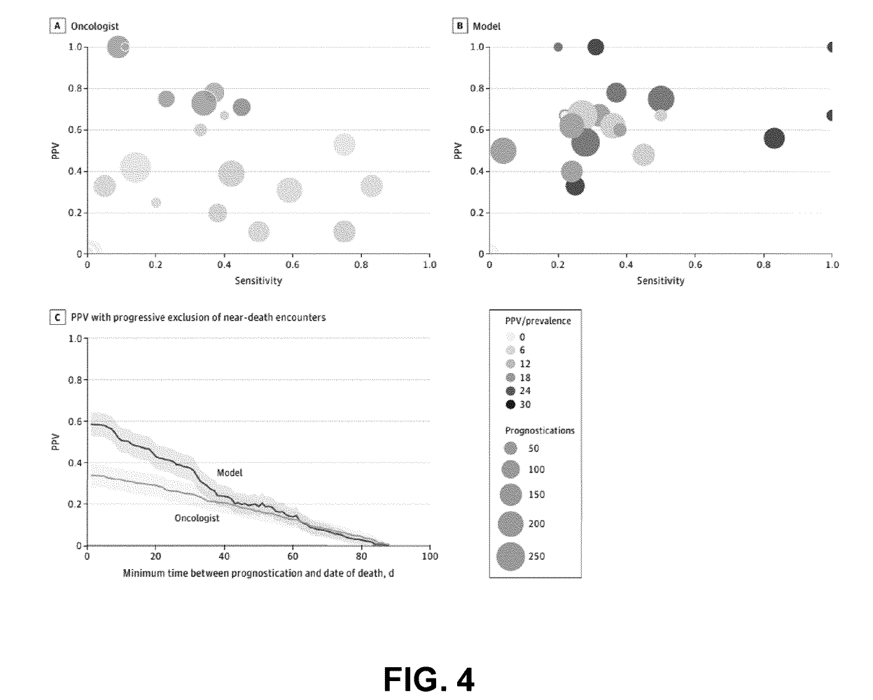

FIG. 4(A) depicts scatterplots showing the positive predictive value (PPV) and sensitivity for the conventional mortality prognostic tool while FIG. 4(B) depicts scatterplots showing the positive predictive value (PPV) and sensitivity of the corresponding predictions made by the mortality prognosis model 115. Dots in the origin indicate instances of exclusively incorrect predictions. The scatterplots of the mortality prognosis model, which shows less scattered points, indicate a higher consistency in the predictions made by the mortality prognosis model 115.

FIG. 4(C) depicts a graph illustrating how the positive predictive value (PPV) for the mortality prognosis model 115 and the conventional mortality prognostic tool varies with progressive exclusion of near-death encounters. For example, where there are no encounters within 36 days from death, the positive predictive values (PPV) of the mortality prognosis model 115 and the conventional mortality prognostic tool would be comparable.

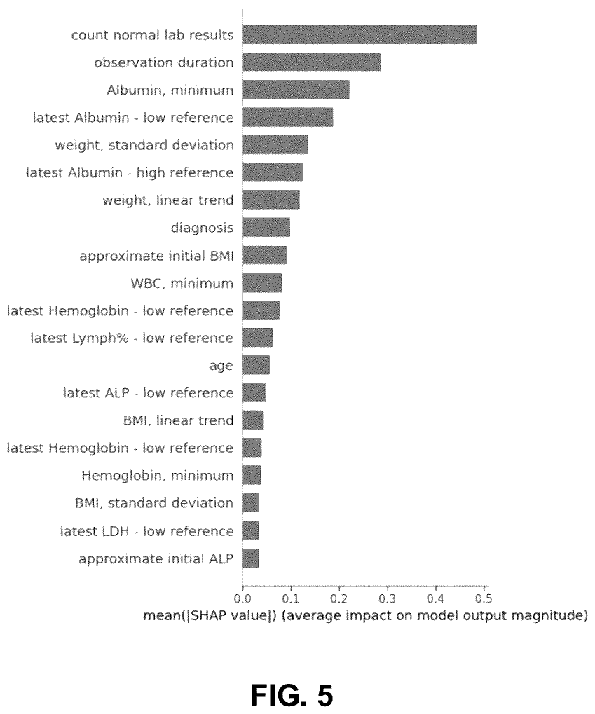

FIG. 5 depicts a bar chart 500 illustrating examples of the most predictive model features over the cohort. As shown in FIG. 5, predictive features used by the mortality prognosis model may include a count of normal lab results, an observation duration, a minimum level of albumin, a low reference for latest albumin level, a standard deviation in weight, a high reference for the latest albumin level, a linear trend in weight, a diagnosis, an approximate initial body mass index (BMI), a minimum white blood cell count (WBC), a low reference for latest hemoglobin level, a low reference for a latest percentage of lymphocytes, age, a low reference for a latest level of alkaline phosphatase (ALP), a linear trend in body mass index (BMI), a minimum level of hemoglobin, a standard deviation in body mass index (BMI), a low reference for latest level of lactate dehydrogenase (LDH), and an approximate initial level of alkaline phosphatase (ALP). In the foregoing, the term “low reference” may refer to a difference between the most recent lab test value and the corresponding low reference value. Lab, weight, and body mass index (BMI) features may be extracted from a particular time window (e.g., six months). The approximate initial body mass index (BMI) may correspond to the intercept of the linear interpolation of body mass index (BMI) time series data.

Because of changes in therapy, some patients may be associated with multiple predictions made by the conventional mortality prognostic tool. For example, in the aforementioned patient cohort, 708 patients had multiple (up to 6) surprise question answers, encompassing 1766 predictions (57.0%). Accordingly, in some cases, the patient cohort may be split between patients with having a single prediction from the conventional mortality prognostic tool and those with multiple predictions from the conventional mortality prognostic tool, with the analysis encompassing all predictions from the conventional mortality prognostic tool. A larger difference in positive predictive value (PPV) was observed between the mortality prognosis model 115 and the conventional mortality prognostic tool for the subpopulation with changes in therapy (PPV difference, 28.4%; 95% CI, 19.7%-37.2%; P<0.001 vs PPV difference, 15.9%; 95% CI, 5.9%-26.0%; P=0.002) (Table 3). Moreover, for this subpopulation, the prediction of the conventional mortality prognostic tool changed for only 31 patients whereas the correct prognosis should have changed for 155 patients.

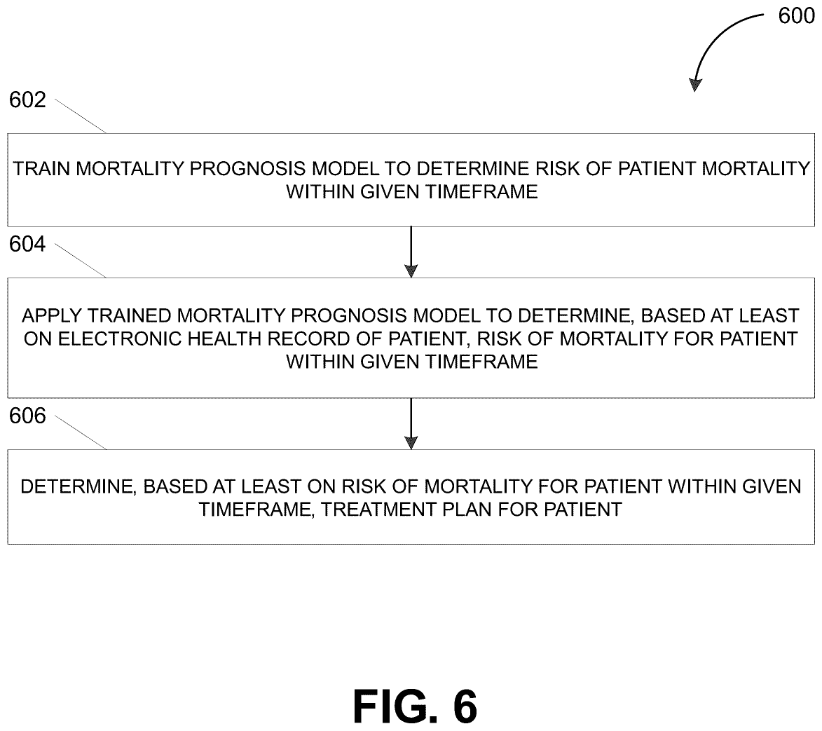

FIG. 6 depicts a flowchart illustrating an example of a process 600 for machine learning enabled prognosis of patient mortality, in accordance with some example embodiments. Referring to FIGS. 1-6, the process 600 may be performed by the prognostic engine 110.

At 602, the prognostic engine 110 may train the mortality prognosis model 115 to determine a risk of patient mortality within a given timeframe. In some example embodiments, the mortality prognosis model 115 may be implemented using one or more machine learning models including, for example, logistic regression, tree ensembles such as random forest and gradient boosted trees, support vector machines, k-nearest neighbors, shallow or deep neural networks and/or the like. The mortality prognosis model 115 may be trained in a supervised manner using a set of labeled training samples. For example, each training sample may be associated with a patient and may include one or more features collected from the patient's electronic health records such as demographic characteristics, laboratory test results, flowsheets, and diagnoses as well as a ground truth annotation of an observation of the patient being deceased or alive at a particular timepoint (e.g., one week, two weeks, three weeks, one month, three months, six months, nine months, one year, 18 months, two years, three years, five years, and/or the like). Training of the mortality prognosis model 115 may include adjusting one or more weights applied by the underlying machine learning model to minimize an error in an output of the mortality prognosis model 115 such that the risk of mortality determined by the mortality prognosis model 115 for a training sample (e.g., the features extracted from a patient's electronic health record (EHR)) corresponds to the ground truth label associated with the training sample (e.g., the observation of whether the patient is deceased or alive at the particular timepoint).

At 604, the prognostic engine 110 may apply the trained mortality prognosis model 115 to determine, based at least on a health record of a patient, a risk of mortality for the patient within the given timeframe. For example, in some example embodiments, the prognostic engine 110 may apply the trained mortality prognosis model 115 to determine, based at least on the electronic health record of a patient, a risk of mortality for the patient within a particular timeframe (e.g., one week, two weeks, three weeks, one month, three months, six months, nine months, one year, 18 months, two years, three years, five years, and/or the like).

At 606, the prognostic engine 110 may determine, based at least on the risk of mortality for the patient within the given timeframe, a treatment plan for the patient. In some example embodiments, the patient's risk of mortality within the particular timeframe (e.g., one week, two weeks, three weeks, one month, three months, six months, nine months, one year, 18 months, two years, three years, five years, and/or the like) may inform treatment decisions including choices for end-of-life care where the patient's risk of mortality satisfies a threshold value. Moreover, in addition to the patient's risk of mortality within one or more timeframes, the patient's treatment plan may be further determined to take into account one or more patient preferences, patient values, and/or patient priorities. In some cases, the patient's treatment plan may include certain options depending on the patient's risk of mortality at different timeframes. For example, the treatment plan for the patient may include clinical trials depending on the patient's risk of mortality at one year or 18 months whereas the treatment plan for the patient may include end-of-life care (e.g., hospice care) depending on the patient's risk of mortality at three months or six months. In some cases, the patient's treatment plan may undergo periodic revaluation. For instance, the trained mortality prognosis model 115 may be applied at different timepoints (e.g., a first timepoint followed by a second timepoint) and the treatment plan for the patient may be adjusted if the patient's risk of mortality determined at these different timepoints exhibit significant (e.g., above-threshold) changes.

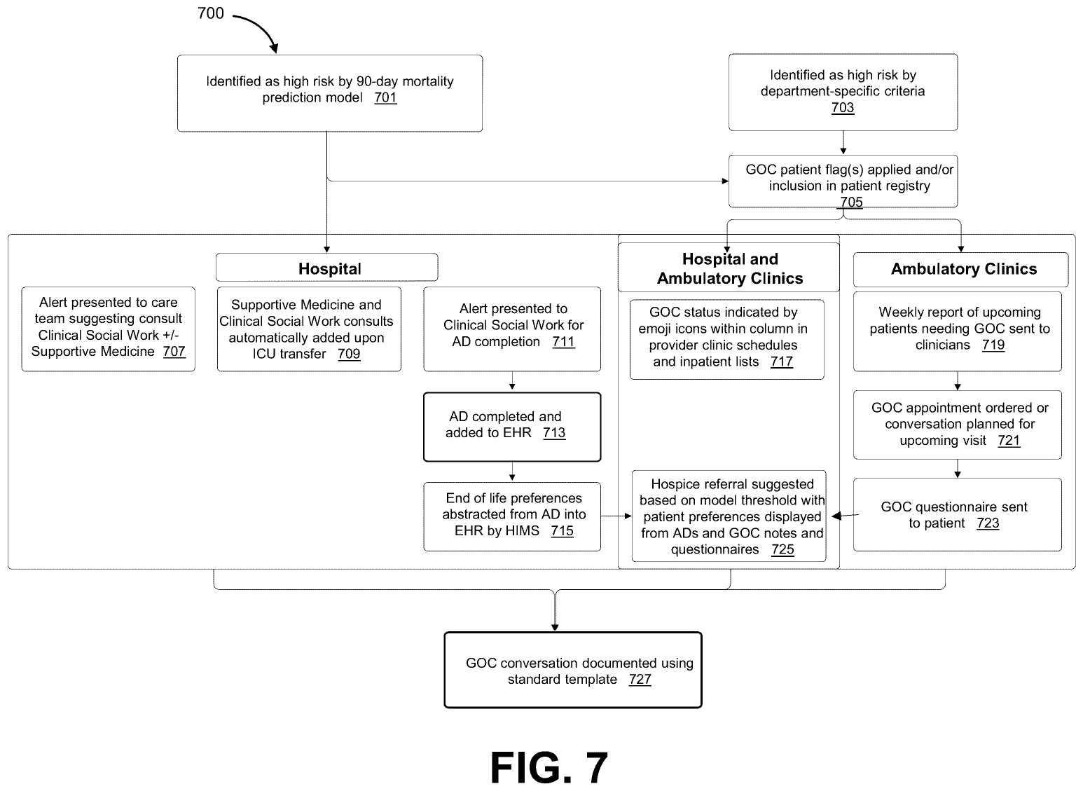

FIG. 7 depicts a block diagram illustrating an example system integrated with a prognosis system. For example a prognosis system or mortality model implementing the machine learning based techniques described herein can be embedded within an operational framework for goal concordant care in a clinical setting. For example, high risk patients can be identified using a mortality prognosis model 115 and/or identification via a health record such as an electronic health record (EHR). Output from the mortality prognosis model 115 can be used to identify high-risk patients and used to trigger processes for completion of goals of care discussions and/or facilitating advance care planning (ACP). In this manner clinical decision support can be facilitated by the prognosis or mortality model.

Process 700 illustrates a concordant care operational framework that utilizes a prognosis or mortality machine learning model such as the trained mortality prognosis model 115. For example, process 700 may include identifying patients as high risk by applying a 90-day mortality prediction model 701 and/or by evaluating patient parameters across department-specific criteria for being high-risk 703.

When a patient is identified as high-risk by the trained mortality prognosis model 115 in step 701 and/or by applying department specific criteria in step 703 information and data from the application of the prognosis model and/or department specific criteria can be recorded in the EHR into a registry database and/or by applying a flag on the patient's record in 705. If the process notes a high-risk patient at step 705, in some embodiments, the system may trigger secondary alerts that can include sending a message (via email or secure portal) to a provider indicating that a goals of care discussion is recommended. One or more of the alerts, setting a flag, or sending of a message may be automated, or automatically transmitted responsive to a determination or output by the trained mortality prognosis model 115.

As illustrated in FIG. 7, the output from the mortality prediction model and/or triggering of the goals of care in step 705 can be implemented into different workflows depending on at least the clinical setting. For example, in a hospital setting, an alert based on a patient being identified as high risk by the 90-day mortality prediction model in 701, and/or based on a patient Goals of Care flag being triggered in 705 can create one or more alerts for the clinical team 707. For example, the alerts for the clinical team 707 can establish communication with clinical social work departments and/or supportive medicine team members to suggest a consult. Additionally, or alternatively, in some embodiments, supportive medicine and clinical social work consults can be automatically added by the concordant care operational framework upon transfer to an intensive care unit in step 709. Further, the system can be configured to transmit an alert to a clinical social worker 711. The transmitted alert 711 can indicate that an advance directive (AD) or POLST: Portable Medical Orders form needs to be completed. The advance directive can be completed and added to an electronic health record (EHR) for the patient in step 713. In some embodiments, end of life preferences can be extracted from an advance directive and inputted into an electronic health record (EHR) at step 715. This process can utilize a health information management system at step 715.

In a hospital or ambulatory care center, goals of care patient flags determined by the machine learning model in step 705 can be indicated via icons, colors, and the like, within electronic schedules and other documents provided to clinicians at step 717. For example, in some embodiments, goals of care status can be indicated by automatically generated emojis which are provided in a column in provider clinic schedules and inpatient lists.

In some ambulatory clinics a weekly report of upcoming patients needing to have goals of care discussions may be generated and communicated to clinicians at step 719. For example, the system may order a goals of care appointment, or issue a notification to request a goals of care appointment 721. In some embodiments, a goals of care questionnaire can be sent to the patient in step 723 prior to or currently with the appointment.

Output from one or more of steps 707, 709, 711, 717, and 719 can be used to guide completion of an advanced directive and goals of care discussions. In some embodiments, the goal concordant care framework can automatically generate hospice referrals based on the model threshold with patient preferences determined and used to generate the referrals. These patient preferences can be determined based on advanced directives and goals of care notes 725.

In some embodiments, a goals of care conversation can be documented at step 727 using a standardized template in all of the described clinical settings.

In some embodiments, one or more of the steps 701, 705, 707, 709, 711, 717, 719, 721, 723, and 725 of the process 700 illustrated in FIG. 7 can be augmented using artificial intelligence and machine learning techniques. Additionally steps 713 and/or 727 can be output from the artificial intelligence-based steps.

In some embodiments, patient data and information corresponding to patients identified as being high risk by the mortality prediction model in 701 can be stored in a patient database or registry 705 that is configured to aggregate patients identified by the mortality prediction model. A goals of care alert, icon or flag can be used to tag patients having entries in such a registry. In some embodiments, clinical decision support can be driven based on polling the registry. For example, in some embodiments, a goals of care flag or inclusion in the patient registry creates a denominator of patients for engagement. Further other criteria such as an absence of advance directives, absence of goals of care, increased acuity level of care by going to the ICU can trigger alerts or initiate downstream actions occur to support goal concordant care delivery.

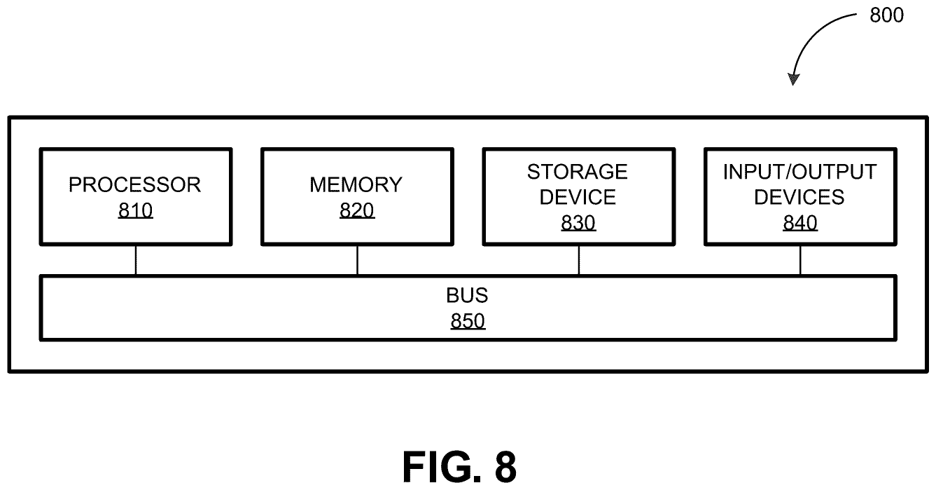

FIG. 8 depicts a block diagram illustrating an example of a computing system 800 consistent with implementations of the current subject matter. Referring to FIGS. 1-8, the computing system 800 can be used to implement the prognostic engine 110 and/or any components therein.

As shown in FIG. 8, the computing system 800 can include a processor 810, a memory 820, a storage device 830, and an input/output device 840. The processor 810, the memory 820, the storage device 830, and the input/output device 840 can be interconnected via a system bus 850. The processor 810 is capable of processing instructions for execution within the computing system 800. Such executed instructions can implement one or more components of, for example, the prognostic engine 110. In some example embodiments, the processor 810 can be a single-threaded processor. Alternately, the processor 810 can be a multi-threaded processor. The processor 810 is capable of processing instructions stored in the memory 820 and/or on the storage device 830 to display graphical information for a user interface provided via the input/output device 840.

The memory 820 is a computer readable medium such as volatile or non-volatile that stores information within the computing system 800. The memory 820 can store data structures representing configuration object databases, for example. The storage device 830 is capable of providing persistent storage for the computing system 800. The storage device 830 can be a solid state drive, a floppy disk device, a hard disk device, an optical disk device, or a tape device, or other suitable persistent storage means. The input/output device 840 provides input/output operations for the computing system 800. In some example embodiments, the input/output device 840 includes a keyboard and/or pointing device. In various implementations, the input/output device 840 includes a display unit for displaying graphical user interfaces.

According to some example embodiments, the input/output device 840 can provide input/output operations for a network device. For example, the input/output device 840 can include Ethernet ports or other networking ports to communicate with one or more wired and/or wireless networks (e.g., a local area network (LAN), a wide area network (WAN), the Internet).

In some example embodiments, the computing system 800 can be used to execute various interactive computer software applications that can be used for organization, analysis and/or storage of data in various formats. Alternatively, the computing system 800 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 840. The user interface can be generated and presented to a user by the computing system 800 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random query memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, recurrent provided to the user can be any form of sensory recurrent, such as for example visual recurrent, auditory recurrent, or tactile recurrent; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

Systems, methods, and articles of manufacture, including computer program products, are provided for machine learning enabled prediction of mortality risks. Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer based methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc. Such computer systems may or may not rely on cloud based architectures.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A computer-implemented method, comprising:
training, based at least on a set of labeled training samples, a mortality prognosis model to determine a risk of patient mortality within a given timeframe;
applying the trained mortality prognosis model to determine, based at least on a health record of a patient, a risk of mortality for the patient within the given timeframe; and
determining, based at least on the risk of mortality for the patient within the given timeframe, a treatment plan for the patient.

2. The method of claim 1, wherein the mortality prognosis model is a machine learning model.

3. The method of claim 1, wherein the mortality prognosis model is based on one or more of a logistic regression, a tree ensemble, a support vector machine, a k-nearest neighbor clustering model, a shallow neural network, or a deep neural network.

4. The method of claim 1, wherein the trained mortality prognosis model determines, based at least on a plurality of features extracted from the health record of the patient, the risk of mortality for the patient within the given timeframe.

5. The method of claim 4, wherein the plurality of features include one or more clinical attributes including at least one of a count of normal lab results, an observation duration, a minimum level of albumin, a low reference for latest albumin level, a standard deviation in weight, a high reference for the latest albumin level, a linear trend in weight, a diagnosis, an approximate initial body mass index (BMI), a minimum white blood cell count (WBC), a low reference for latest hemoglobin level, a low reference for a latest percentage of lymphocytes, age, a low reference for a latest level of alkaline phosphatase (ALP), a linear trend in body mass index (BMI), a minimum level of hemoglobin, a standard deviation in body mass index (BMI), a low reference for latest level of lactate dehydrogenase (LDH), an approximate initial level of alkaline phosphatase (ALP), a diagnosis, a medication, a performance status, utilization, notes, and Logical Observation Identifiers Names and Codes (LOINC).

6. The method of claim 1, wherein the health record is an electronic health record of the patient.

7. The method of claim 1, wherein the set of labeled training samples include a plurality of training samples, and wherein each training sample of the plurality of training samples is associated with a patient.

8. The method of claim 7, wherein each training sample of the plurality of training samples include one or more demographic characteristics, laboratory test results, flowsheets, and diagnoses of a corresponding patient.

9. The method of claim 1, wherein one or more training samples of the plurality of training samples further include a ground truth annotation of an observation of the corresponding patient being deceased or alive at an end of the given timeframe.

10. The method of claim 1, further comprising:
generating the set of labeled training samples to include no more than a threshold quantity of training samples associated with patients who die within a threshold quantity of time.

11. The method of claim 1, wherein the given timeframe is one week, two weeks, three weeks, one month, three months, six months, nine months, one year, 18 months, two years, three years, or five years.

12. The method of claim 1, wherein the treatment plan for the patient is determined to include end-of-life care where the risk of mortality for the patient satisfies a threshold value.

13. The method of claim 1, wherein the treatment plan for the patient is further determined based on one or more patient preferences, patient values, and patient priorities.

14. The method of claim 1, wherein the treatment plan for the patient is further determined based on one or more patient preferences, patient values, and patient priorities where the risk of mortality for the patient satisfies a threshold value.

15. The method of claim 1, wherein the treatment plan for the patient is determined to include one or more clinical trials where the risk of mortality for the patient satisfies a threshold value.

16. The method of claim 1, wherein the treatment plan for the patient is determined to include end-of-life care where the risk of mortality for the patient within a first timeframe satisfies a first threshold value, and wherein the treatment plan for the patient is determined to include one or more clinical trials where the risk of mortality for the patient within the first timeframe and/or a second timeframe satisfies a second threshold value.

17. The method of claim 1, further comprising:
revaluating the treatment plan for the patient where the risk of mortality for the patient satisfies a threshold value.

18. The method of claim 1, wherein the revaluating of the treatment plan includes applying the trained mortality prognosis model to determine the risk of mortality for the patient at a first timepoint before applying the trained mortality prognosis model to determine the risk of mortality for the patient at a second timepoint, and adjusting the treatment plan where a difference in the risk of mortality for the patient at the first timepoint and the second timepoint exceeds a threshold value.

19. A system, comprising:
at least one data processor; and
at least one memory storing instructions, which when executed by the at least one data processor, result in operations comprising:
training, based at least on a set of labeled training samples, a mortality prognosis model to determine a risk of patient mortality within a given timeframe;
applying the trained mortality prognosis model to determine, based at least on a health record of a patient, a risk of mortality for the patient within the given timeframe; and
determining, based at least on the risk of mortality for the patient within the given timeframe, a treatment plan for the patient.

20. A non-transitory computer readable medium storing instructions, which when executed by at least one data processor, result in operations comprising:
training, based at least on a set of labeled training samples, a mortality prognosis model to determine a risk of patient mortality within a given timeframe;
applying the trained mortality prognosis model to determine, based at least on a health record of a patient, a risk of mortality for the patient within the given timeframe; and
determining, based at least on the risk of mortality for the patient within the given timeframe, a treatment plan for the patient.
